(19) United States
(12) Patent Application Publication
(10) Pub. No.: US 2023/0191042 A1
(43) Pub. Date: Jun. 22, 2023

(54) BLOOD VESSEL VISUALIZATION APPARATUS AND BLOOD VESSEL PUNCTURE SYSTEM

(71) Applicant: TERUMO KABUSHIKI KAISHA, Tokyo (JP)

(72) Inventors: Shota YAMAMOTO, Mitaka-shi (JP); Takayuki YOKOTA, Chuo-shi (JP)

(73) Assignee: TERUMO KABUSHIKI KAISHA, Tokyo (JP)

(21) Appl. No.: 18/108,183

(22) Filed: Feb. 10, 2023

Related U.S. Application Data
(63) Continuation of application No. PCT/JP2021/030493, filed on Aug. 20, 2021.

(30) Foreign Application Priority Data
Aug. 26, 2020 (JP) 2020-142276

(51) Int. Cl. A61M 5/42

(52) U.S. Cl. CPC A61M 5/427; A61M 2025/0166

(57) ABSTRACT

A blood vessel visualization apparatus for visualizing a blood vessel inside a living body with light that has been transmitted through the living body includes: an irradiation unit configured to emit light from a contact portion with skin of the living body, the irradiation unit including: a fixing portion configured to be fixed to a target site of the living body, wherein the fixing portion includes a surface configured to contact the skin of the living body, and a plurality of light sources located in a light source arrangement area at the surface of the fixing portion that is configured to contact the skin of the living body. A brightness and/or a light distribution angle of respective ones of the light sources differ according to positions of the light sources in the light source arrangement area.

### CROSS-REFERENCE TO RELATED APPLICATIONS

This is a bypass continuation of PCT Application No. PCT/JP2021/030493, filed on Aug. 20, 2021, which claims priority to Japanese Application No. JP2020-142276, filed on Aug. 26, 2020. The contents of these applications are incorporated by reference in their entireties.

### BACKGROUND

The present invention relates to a blood vessel visualization apparatus and a blood vessel puncture system that irradiate a living body with light and visualize blood vessels.

There has been proposed a blood vessel visualization apparatus that irradiates a target site of a living body with light and visualizes a blood vessel with transmitted light of a target site. For example, JP 2018-64666 A discloses a vein visualization apparatus that can irradiate an arm with visible light from light emitting elements by winding and arranging around and on the arm a flexible plate member on which the plurality of light emitting elements are arranged, and visualize a vein.

### SUMMARY

An apparatus that visualizes a blood vessel of a living body by transmitted light desirably makes an image of the blood vessel of a target site appear under the transmitted light of a uniform luminance. However, for example, the target site of the living body such as an arm or a foot does not have a uniform thickness, and therefore there is a problem that local brightness and darkness are likely to occur, the luminance locally varies, and it becomes difficult to distinguish the blood vessel.

Furthermore, in a case in which an image of transmitted light is captured and an image processing apparatus visualizes the blood vessel, when an image of a target site has variations in a local luminance variation, it is difficult to perform the image processing.

Furthermore, arranging light sources in a wide area as in JP 2018-64666 A to reduce variations in a luminance of a target site increases light that goes around surroundings of the target site and reaches the imaging element, and thereby causes a problem that transmitted light near the center of the target site is cancelled out, and a blood vessel becomes difficult to see.

An object of the present invention is to provide a blood vessel visualization apparatus and a blood vessel puncture system capable of visualizing an image of a blood vessel of a target site under a uniform luminance.

One aspect of the following disclosure is a blood vessel visualization apparatus is a blood vessel visualization apparatus that includes an irradiation unit that emits light from a contact portion with a skin of a living body, and visualizes a blood vessel inside the living body with transmitted light having transmitted through the living body, the irradiation unit includes a fixing portion that is fixed to a target site of the living body and is in contact with the skin of the living body, a light source arrangement area that is provided in the fixing portion and provided at a portion in contact with the skin of the living body, and a plurality of light sources that are arranged in the light source arrangement area, and one or both of brightness and a light distribution angle of each of the plurality of light sources differ according to a position in the light source arrangement area.

Another aspect is a blood vessel puncture system that includes: the blood vessel visualization apparatus according to the above aspect; and a puncture needle or a catheter assembly.

The blood vessel visualization apparatus and the blood vessel puncture system according to the above aspect can visualize an image of a blood vessel of a target site under a uniform luminance.

### BRIEF DESCRIPTION OF DRAWINGS

FIG. 1 is a diagram illustrating a configuration of a blood vessel visualization apparatus according to a first embodiment;

FIG. 2 is a perspective view of a state where an irradiation unit in FIG. 1 is attached to a target site (arm part) of a living body;

FIG. 3 is a schematic view illustrating cross sections of the irradiation unit and the target site, a light reception unit, and a display unit along a line III-III (A direction) in FIG. 2;

FIG. 4A is a view illustrating a relationship between a position in the A direction in FIG. 3 and a thickness of the target site, and FIG. 4B is a view illustrating a relationship between the position in the A direction in FIG. 3 and brightness of a light source;

FIG. 5A is a cross-sectional schematic view taken along a line VA-VA (B direction) in FIG. 2, and FIG. 5B is a view illustrating a relationship between a position in the B direction in FIG. 5A and the brightness of the light source;

FIG. 6 is a cross-sectional view illustrating an arrangement example of a light source along the direction A in FIG. 2 according to a modified example of the first embodiment;

FIG. 7A is a cross-sectional schematic view illustrating a blood vessel visualization apparatus according to a second embodiment, and FIG. 7B is a cross-sectional schematic view illustrating a blood vessel visualization apparatus according to a modified example of the second embodiment;

FIG. 8A is a side view of a light source according to a third embodiment, and FIG. 8B is a cross-sectional schematic view of a state where an irradiation unit of the blood vessel visualization apparatus according to the third embodiment that uses the light sources in FIG. 8A is attached to a target site of a living body; and

FIG. 9 is a schematic view illustrating a cross section of an irradiation unit, a light reception unit, and a display unit of a blood vessel puncture system according to a fourth embodiment.

### DETAILED DESCRIPTION

Hereinafter, preferred embodiments of the present invention will be described in detail with reference to the accompanying drawings. Note that dimensional ratios in the drawings may be exaggerated and different from actual ratios for convenience of description.

### First Embodiment

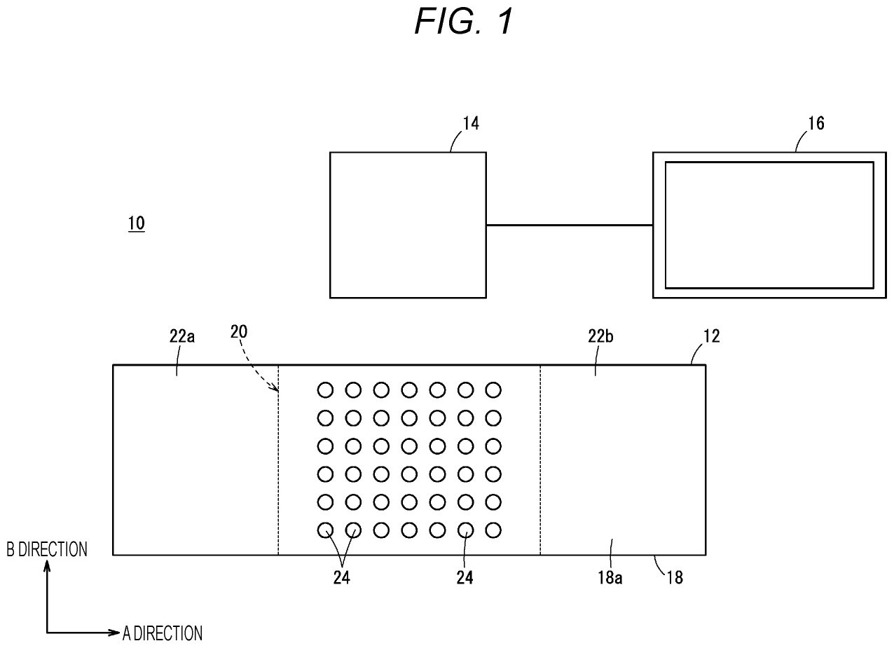

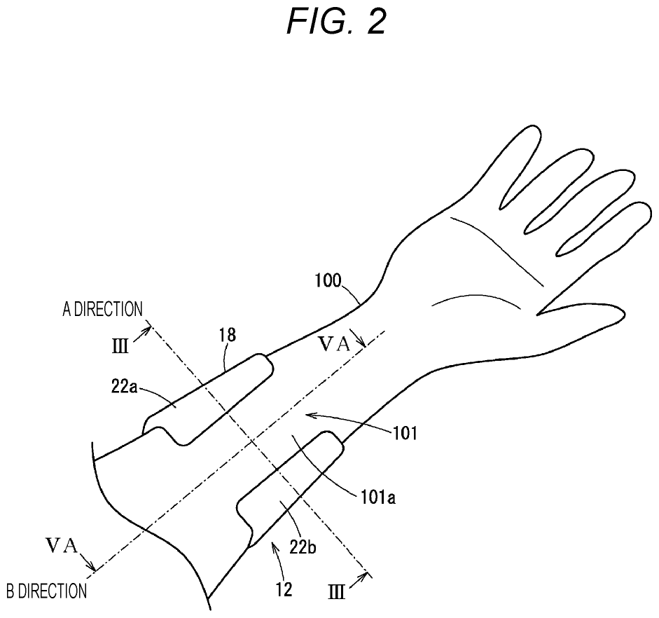

As illustrated in FIG. 1, a blood vessel visualization apparatus 10 includes an irradiation unit 12, a light reception unit 14, and a display unit 16. The irradiation unit 12 includes a plate fixing portion 18 that has flexibility. The fixing portion 18 is formed in a rectangular shape elongated in an A direction in the drawing. A light source arrangement area 20 is provided at a center portion of a surface 18a of the fixing portion 18 in the A direction. Furthermore, abutting portions 22a and 22b are respectively provided on both side portions of the light source arrangement area 20 in the A direction. As illustrated in FIG. 2, the fixing portion 18 can be bent to match a shape of a target site 101 (an arm part in the illustrated example) of a living body 100. When wound around the target site 101, the abutting portions 22a and 22b keep the shapes. Furthermore, the fixing portion 18 is fixed by being wound around the target site 101. At this time, the surface 18a of the fixing portion 18 comes into contact with the target site 101.

As illustrated in FIG. 1, a plurality of light sources 24 are arranged in the light source arrangement area 20 on the surface 18a. The light sources 24 are, for example, light emitting elements such as light emitting diodes or EL light emitting elements, and are two-dimensionally and regularly arranged in the light source arrangement area 20. The dimension of the light source arrangement area 20 in a B direction can be the same as the dimension of the fixing portion 18 in the B direction.

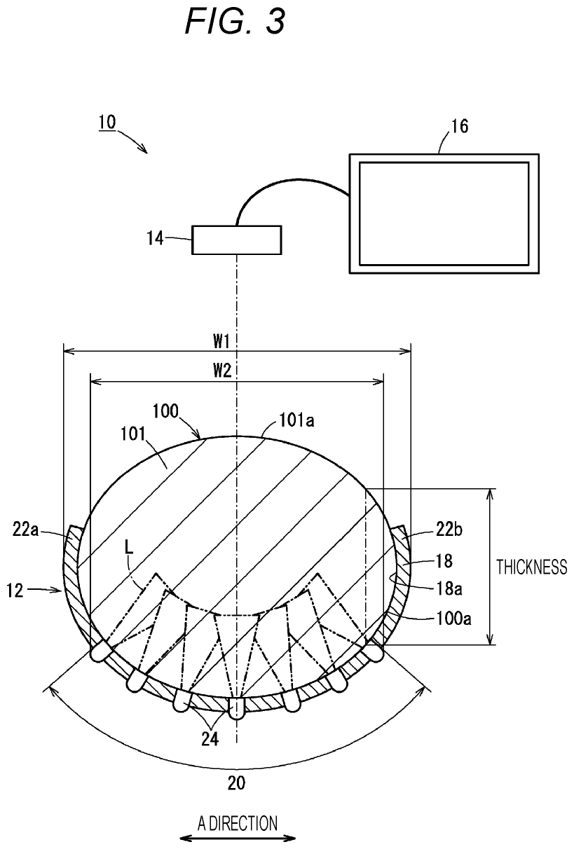

Furthermore, as illustrated in FIG. 3, the dimension of the light source arrangement area 20 in the A direction is set such that, when the fixing portion 18 is wound around the target site 101 of the living body 100, a width W2 occupied by the light source arrangement area 20 is approximately 40% to 80% of a total width W1 of a wound portion. A specific value of the width W2 can be appropriately adjusted according to the optical characteristics (a light distribution angle and a luminance) of the light source 24 within the above range. In addition, when the width W2 occupied by the light source arrangement area 20 becomes smaller than the above rate, the target site 101 cannot be illuminated with uniform brightness, and the luminance varies. Furthermore, when the rate of the width W2 occupied by the light source arrangement area 20 is made larger than the above value, the light going around a side portion of the target site 101 increases, and visibility of blood vessels of the target site 101 lowers.

The light source 24 may be formed by bonding a light emitting element (e.g., light emitting diode) as a discrete part to the fixing portion 18. Furthermore, the light sources 24 may be formed by attaching a light emitting element that is a module part formed by planarly arranging and integrating the plurality of light sources 24 to the light source arrangement area 20 of the fixing portion 18. As described later, the light source 24 has respectively different light irradiation characteristics depending on a position in the light source arrangement area 20. In the present embodiment, the light distribution angle (irradiation angle range L) of each light source 24 in the light source arrangement area 20 is fixed.

As the light source 24, for example, an infrared light emitting diode that emits near infrared light whose center wavelength is, for example, 700 nm or more and 2500 nm or less, preferably 700 nm or more and 1400 nm or less, and more preferably 780 nm or more and 940 nm or less can be used. Furthermore, the light source 24 may emit visible light (that does not include near infrared light). Furthermore, the light source 24 may emit light including both of near infrared light and visible light.

For example, a CMOS camera or a CCD camera for visible light or near infrared light is used as the light reception unit 14. As illustrated in FIG. 3, the light reception unit 14 is arranged on an opposite side to and faces the light source arrangement area 20 of the irradiation unit 12 with the target site 101 of the living body 100 interposed therebetween. The light reception unit 14 is a camera (image capturing unit) that captures an image of the target site 101 based on transmitted light of the light sources 24 transmitting through the target site 101 of the living body 100.

The display unit 16 displays the image of the target site 101 obtained by the light reception unit 14 on a display screen.

Next, a shape of the target site 101 of the living body 100 and the light irradiation characteristics of the light sources 24 will be described. The target site 101 of the living body 100 is, for example, a human arm as illustrated in FIG. 2, and can be considered as an elliptical shape having a wide cross section. As illustrated in FIG. 3, considering the cross section in the A direction, the light source 24 emits light at a fixed light distribution angle (irradiation angle range L). The light from the light source 24 passes through the target site 101 toward a direction of the light reception unit 14 while being scattered in the target site 101 of the living body 100, and then passes through the surface 101a of the target site 101.

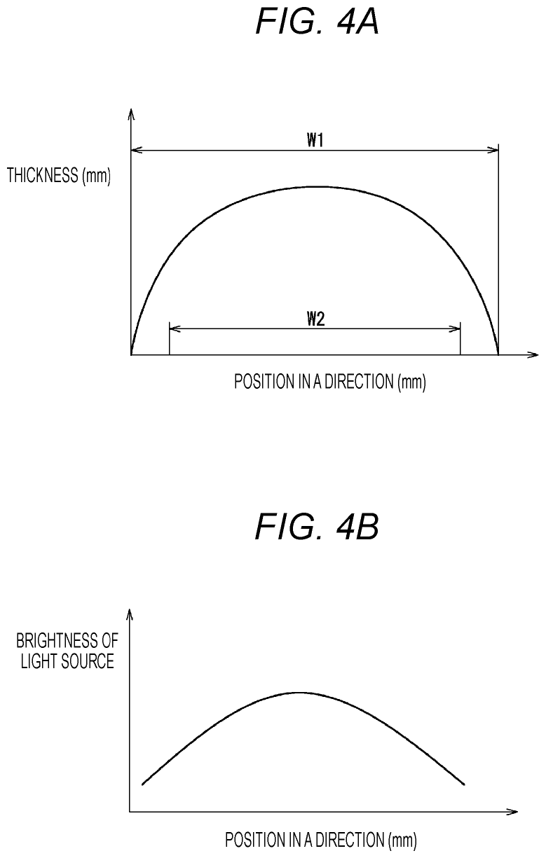

The light emitted from each light source 24 generally passes through the target site 101 in a direction parallel to a line that connects the light source 24 at the center of the light source arrangement area 20 in the direction A, and the light reception unit 14. Therefore, the thickness through which the light passes inside the target site 101 varies depending on the position of the light source 24 in the light source arrangement area 20. In a case in which the shape of the target site 101 is an elliptical shape as illustrated in FIG. 4A, the thickness through which the light emitted from the light source 24 passes inside the target site 101 varies in a distributed manner in the A direction as illustrated in FIG. 4A. When the thickness through which light passes inside the target site 101 increases, the quantity of light absorbed inside the living body 100 also increases, and therefore luminance varies.

Hence, in the present embodiment, the brightness (luminous flux [lm]) of each light source 24 varies in FIG. 4B. That is, near a periphery portion of the light source arrangement area 20 in the A direction at which the thickness through which irradiation light passes inside the target site 101 decreases, the brightness of the light source 24 is set relatively low. Furthermore, near a center portion of the light source arrangement area 20 in the A direction at which the thickness through which the irradiation light passes inside the target site 101 increases, the brightness of the light source 24 is set relatively high.

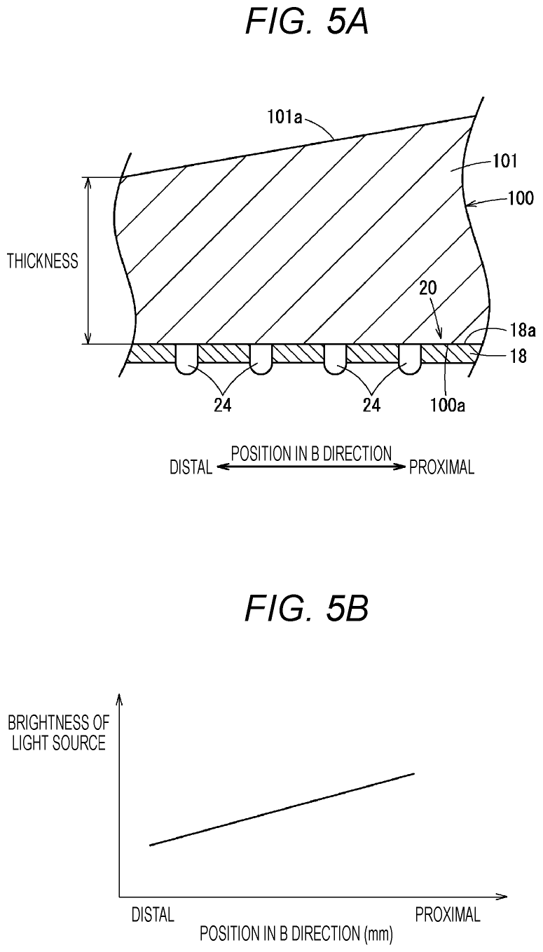

Furthermore, in a case in which the thickness is different between a distal side and a proximal side (similar to the case in which the target site 101 is the arm as illustrated in FIG. 5A), the thickness through which the irradiation light passes inside the target site 101 varies according to the position in the light source arrangement area 20 in the B direction. Hence, in the present embodiment, as illustrated in FIG. 5B, the brightness of the light source 24 may be smaller on the distal side of the light source arrangement area 20, and may be larger on the proximal side of the light source arrangement area 20. Note that, depending on the shape of the target site 101, the distribution of the brightness of the light source 24 in the B direction may be reversed from the above, or the brightness of the light sources 24 may not be differed in the B direction.

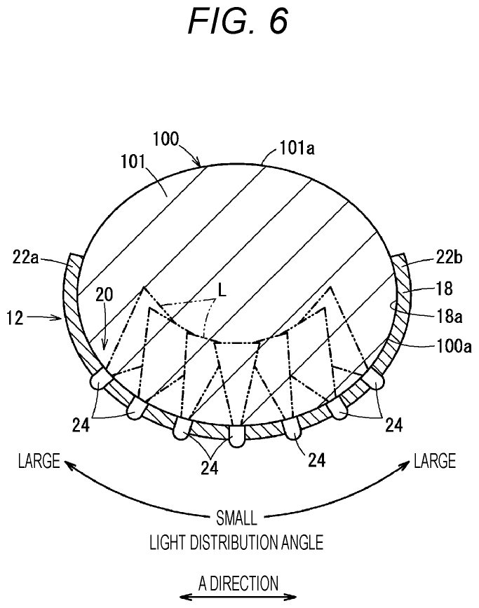

Note that, in the present embodiment, as illustrated in FIG. 6, instead of changing the brightness (luminous flux [lm]) of the light source 24, the light distribution angle (irradiation angle [sr]) of the light source 24 in the light source arrangement area 20 may vary. That is, near the periphery portion of the light source arrangement area 20 in the A direction at which the thickness through which the irradiation light passes inside the target site 101 decreases, the light distribution angle of the light source 24 is set large. Furthermore, near the center portion of the light source arrangement area 20 in the A direction at which the thickness through which the irradiation light passes inside the target site 101 increases, the light distribution angle of the light source 24 is set small.

Furthermore, instead of determining the distribution of the brightness of each light source 24 of the light source arrangement area 20 according to the model having an elliptical cross section as described above, the distribution of the brightness may be set such that the luminance value of the transmitted light is constant on the surface 101a of the target site 101 facing the light reception unit 14. In this case, there may be employed a configuration where a luminance distribution of the image of the light reception unit 14 may be obtained, and an unillustrated control unit individually sets the brightness or the light distribution angle of the light source 24 under a condition that the luminance distribution is uniform.

The blood vessel visualization apparatus 10 according to the present embodiment has the following effects.

The blood vessel visualization apparatus 10 according to the present embodiment is the blood vessel visualization apparatus 10 that includes the irradiation unit 12 that emits light from the contact portion with the skin 100a of the living body 100, and visualizes blood vessels inside the living body 100 with transmitted light having transmitted through the living body 100. The irradiation unit 12 includes the fixing portion 18 that is fixed to the target site 101 of the living body 100, and is in contact with the skin 100a of the living body 100. The light source arrangement area 20 is provided at the portion of the fixing portion 18 that is in contact with the skin 100a of the living body 100. The light source arrangement area 20 is provided with the plurality of light sources 24, and one or both of the brightness or the light distribution angle of the plurality of these light sources 24 differ according to the position in the light source arrangement area 20.

According to the above configuration, it is possible to vary the brightness or the light distribution angle of the light source 24 between a portion of large thickness and a portion of small thickness at which the irradiation light passes inside the target site 101 of the living body 100. Consequently, it is possible to make the quantity of light that transmits through the target site 101 uniform, and suppress variations in the luminance of the surface 101a.

In the above blood vessel visualization apparatus 10, the brightness or the light distribution angle of the light sources 24 may vary according to the thickness of the living body 100 along the direction parallel to the line that connects the light source 24 at the center of the light source arrangement area 20 and the light reception unit 14. According to this configuration, it is possible to prevent the variations in the luminance on the surface 101a of the target site 101. In this case, the living body 100 may be a columnar body having an elliptical cross section, and the brightness or the light distribution angle of the light source 24 may be obtained.

In the above blood vessel visualization apparatus 10, the brightness or the light distribution angle of each of the plurality of light sources 24 may be set such that the illuminance distribution of the transmitted light to the surface 101a of the target site 101 of the living body 100 is uniform. According to this configuration, it is possible to suppress the variations in the luminance of the target site 101. In this case, the brightness or the light distribution angle of the light source 24 may be configured to be individually adjusted under control of the control unit based on the captured image of the light reception unit 14.

In the above blood vessel visualization apparatus 10, the fixing portion 18 is formed in the shape that is elongated in the direction crossing the target site 101 of the living body 100, three or more of the light sources 24 are arrayed along the longitudinal axis of the fixing portion 18, and the light source 24 located at the center among the light sources 24 has the highest brightness. According to this configuration, by maximizing the brightness of the light source 24 at the portion at which the thickness through which the irradiation light passes inside the target site 101 of the living body 100 is the largest, it is possible to suppress the variations in the luminance of the surface 101a of the target site 101.

In the above blood vessel visualization apparatus 10, the fixing portion 18 may be formed in the shape that is elongated in the direction crossing the target site 101 of the living body 100, the three or more light sources 24 may be arrayed along the longitudinal axis of the fixing portion 18, and the light distribution angle of the light source 24 located on an outer side of the longitudinal direction may be larger than the light distribution angle of the light source 24 arranged at the center in the longitudinal direction. According to this configuration, by widening the light distribution angle of the light source 24 on the outer side at which the thickness through which the irradiation light passes inside the target site 101 of the living body 100 becomes thin, it is possible to suppress the variations in the luminance of the surface 101a of the target site 101.

In the above blood vessel visualization apparatus 10, the fixing portion 18 may be deformable according to the shape of the target site 101 of the living body 100. According to this configuration, the fixing portion 18 can be fixed by being wound around the target site 101.

The above blood vessel visualization apparatus 10 may be provided with the light reception unit 14 that captures an image of the transmitted light appearing on the surface 101a of the target site 101 of the living body 100. According to this configuration, light other than visible light can be used as the transmitted light. Furthermore, a transmission image of the living body 100 can be obtained even by weak transmitted light that cannot be visually checked by naked eyes. Furthermore, this configuration may include the display unit 16 that displays an image captured by the light reception unit 14. The display unit 16 makes it possible to visually check the image captured by the light reception unit 14 on the spot.

In the above blood vessel visualization apparatus 10, the irradiation unit 12 and the light reception unit 14 may be arranged to face each other with the target site 101 of the living body 100 interposed therebetween. According to this configuration, the light reception unit 14 captures the transmitted light passing through the target site 101 of the living body 100, so that it is possible to visualize blood vessels.

In the above blood vessel visualization apparatus 10, the light source 24 may emit near infrared light. According to this configuration, it is possible to obtain a clearer visualized image of blood vessels by using near infrared light having high transmissivity with respect to the living body 100.

In the above blood vessel visualization apparatus 10, the wavelength of the near infrared light emitted by the light source 24 may be 700 nm or more and 2500 nm or less, preferably 700 nm or more and 1400 nm or less, and more preferably 780 nm or more and 940 nm or less. Blood has a higher absorption rate of the near infrared light of this wavelength than that of a living tissue (target site), so that the near infrared light is suitable for visualization of blood vessels.

### Second Embodiment

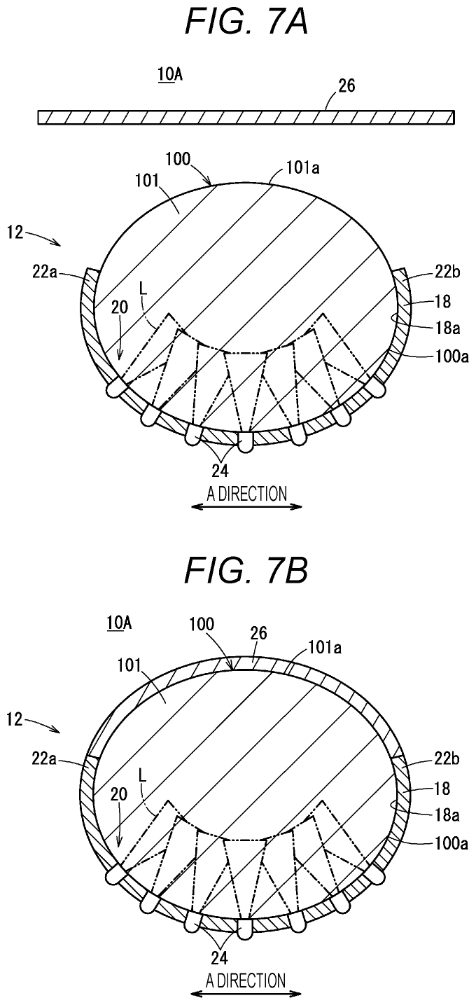

As illustrated in FIG. 7A, a blood vessel visualization apparatus 10A according to the present embodiment includes a light emitter 26 instead of a light reception unit 14 and a display unit 16. The light emitter 26 is a plate member that is formed by applying or impregnating a phosphor to a transparent plate substrate such as glass. The near infrared light having transmitted through living tissues is converted into visible light having a specific wavelength suitable for imaging, and emitted. The light emitter 26 is arranged near the living body 100 so as to face the irradiation unit 12 with the living body 100 interposed therebetween.

By emitting visible light of a specific wavelength having a luminance matching an intensity of the near infrared light having transmitted through the living body 100, the light emitter 26 can visualize blood vessels near a skin 100a of the living body 100.

In addition, as illustrated in FIG. 7B, the light emitter 26 may be arranged by being wound along the surface 101a of the target site 101.

The blood vessel visualization apparatus 10A according to the present embodiment includes the light emitter 26 that converts transmitted light (near infrared light) appearing on the surface 101a of the target site 101 of the living body 100 into light (visible light) of a different wavelength and projects the light. Consequently, even when the near infrared light is used, the light reception unit 14 and the display unit 16 are unnecessary, so that it is possible to simplify the apparatus configuration.

### Third Embodiment

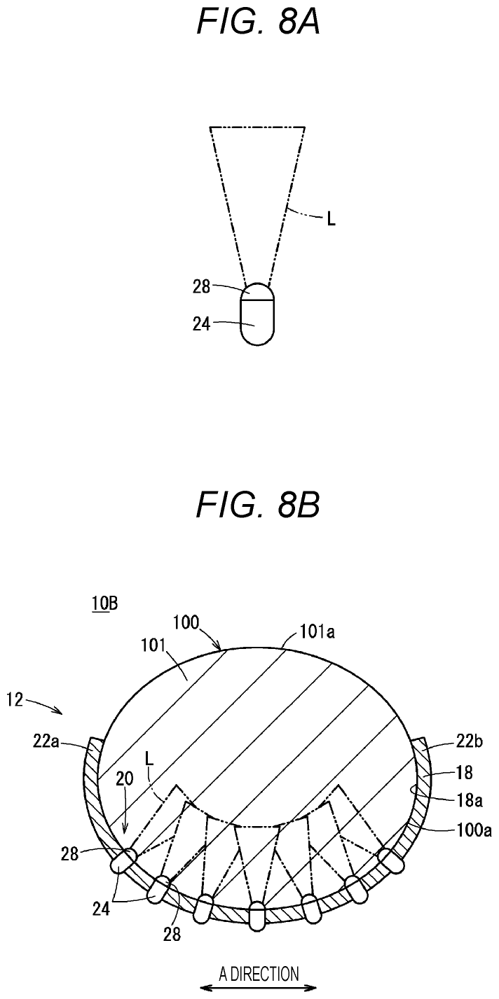

As illustrated in FIGS. 8A and 8B, a blood vessel visualization apparatus 10B according to the present embodiment is provided with a transparent protrusion portion 28 at a contact site of a light source 24 of an irradiation unit 12 with a living body 100. In this regard, the same components of the blood vessel visualization apparatus 10B according to the present embodiment as those of a blood vessel visualization apparatus 10 according to the first embodiment will be assigned the same reference numerals, and the detailed description thereof will be omitted. Furthermore, in FIG. 8B, a light reception unit 14 and a display unit 16 are not illustrated.

The shape of the protrusion portion 28 is not limited in particular, yet is formed in a hemispherical shape having the same diameter as the diameter of the light source 24. The protrusion portion 28 is formed by a flexible material whose refractive index with respect to a wavelength of irradiation light of the light source 24 is equal to a refractive index of a living tissue (target site) at this wavelength. When the irradiation light of the light source 24 is emitted as near infrared light, the protrusion portion 28 can be formed by a flexible silicone resin. Furthermore, the refractive index of the protrusion portion 28 is close to the refractive index of the living body 100, so that it is possible to suppress reflection on a skin 100a of the living body 100.

When a fixing portion 18 is attached to a target site 101 of the living body 100 as illustrated in FIG. 8B, the skin 100a of the living body 100 is deformed into a recessed shape by the protrusion portion 28, and the protrusion portion 28 is pushed into the target site 101. Consequently, the target site 101 of the living body 100 can be more efficiently irradiated with light of the light source 24. Furthermore, light going around the target site 101 decreases, so that it is possible to reduce a noise component that cancels out the transmitted light, and obtain a clearer visualized image of blood vessels.

In the above blood vessel visualization apparatus 10B, the light source 24 includes the protrusion portion 28 at a portion that abuts on the skin 100a of the living body 100. According to this configuration, light efficiently propagates from the portion at which the protrusion portion 28 is pushed into the living body 100. Furthermore, it is possible to suppress generation of light that goes around the target site 101. Furthermore, as the protrusion portion 28 is pushed more, the thickness through which the light passes inside the target site 101 decreases more, so that it is possible to obtain a clearer perspective image.

In the above blood vessel visualization apparatus 10B, the protrusion portion 28 may be made of a soft material that allows light to transmit through. According to this configuration, it is possible to obtain a clearer visualized image of blood vessels.

### Fourth Embodiment

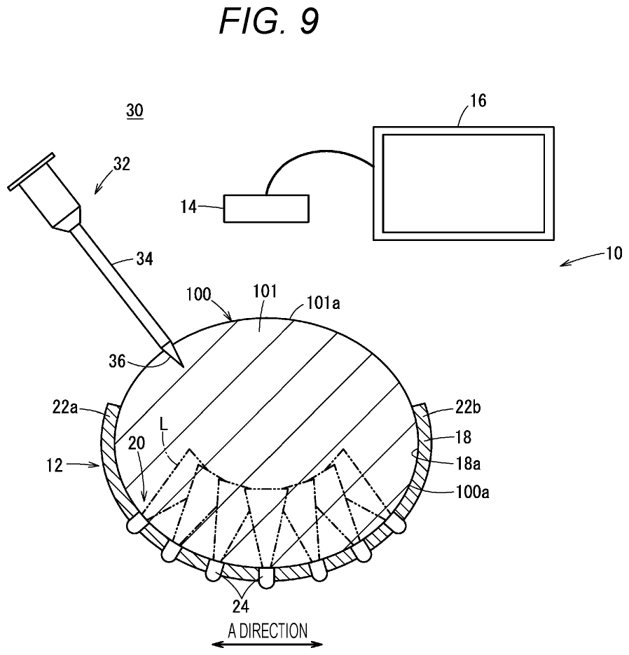

The present embodiment will describe a blood vessel puncture system 30. As illustrated in FIG. 9, the blood vessel puncture system 30 includes a blood vessel visualization apparatus 10 in FIG. 1 and a needle module 32. Hereafter, description of the blood vessel visualization apparatus 10 will be omitted.

The needle module 32 is, for example, an indwelling needle or a catheter assembly placed in a blood vessel. The needle module 32 includes a needle tube 34 and a sharp needle tip 36 located at a distal end of the needle tube 34. The needle tube 34 is made of, for example, a metal material such as stainless steel, and is made of a material having a low transmittance with respect to near infrared light and infrared light. Therefore, a display unit 16 displays the needle tube 34 of the needle module 32 as a black portion.

The needle module 32 is arranged in a space between the light reception unit 14 and a target site 101 of a living body 100. A user can perform a procedure of puncturing a blood vessel while the light reception unit 14 captures a perspective image of the target site 101 of the living body 100.

As described above, the blood vessel puncture system 30 according to the present embodiment includes the blood vessel visualization apparatus 10, and a puncture needle or the catheter assembly (needle module 32).

According to the above configuration, it is possible to make the puncture needle or the catheter assembly puncture a blood vessel while accurately grasping a position of the blood vessel using the blood vessel visualization apparatus 10. The blood vessel visualization apparatus 10 can visualize a wide range with a uniform luminance, so that it is easy to search for a blood vessel suitable for puncturing. Consequently, the blood vessel puncture system 30 can make the puncture needle or the catheter assembly easily puncture a blood vessel.

Although the present invention has been described above citing the preferred embodiments, the present invention is not limited to the above embodiments, and it goes without saying that various modifications can be made without departing from the gist of the present invention.

What is claimed is:

1. A blood vessel visualization apparatus for visualizing a blood vessel inside a living body with light that has been transmitted through the living body, the blood vessel visualization apparatus comprising:
an irradiation unit configured to emit light from a contact portion with skin of the living body, the irradiation unit comprising:
a fixing portion configured to be fixed to a target site of the living body, wherein the fixing portion includes a surface configured to contact the skin of the living body, and
a plurality of light sources located in a light source arrangement area at the surface of the fixing portion that is configured to contact the skin of the living body, wherein:
a brightness and/or a light distribution angle of respective ones of the light sources differ according to positions of the light sources in the light source arrangement area.

2. The blood vessel visualization apparatus according to claim 1, wherein:
the brightness and/or the light distribution angle of each respective one of the light sources vary according to a thickness of the living body along a line extending from the respective one of the light sources in a direction parallel to a line that connects a light reception unit to a centermost one of the light sources in the light source arrangement area.

3. The blood vessel visualization apparatus according to claim 1, wherein:
the brightness and/or the light distribution angle of each of the plurality of light sources is set such that an illuminance distribution of the transmitted light to a surface of the target site of the living body is uniform.

4. The blood vessel visualization apparatus according to claim 1, wherein:
the fixing portion is formed in a shape that is elongated in a direction crossing the target site of the living body, three or more of the light sources are arrayed along a longitudinal axis of the fixing portion, and a brightness of a centermost one of the three or more light sources is highest among the three or more light sources.

5. The blood vessel visualization apparatus according to claim 1, wherein:
the fixing portion is deformable according to a shape of the target site of the living body.

6. The blood vessel visualization apparatus according to claim 5, wherein:
the fixing portion is fixed to the skin of the living body by being wound around the target site of the living body.

7. The blood vessel visualization apparatus according to claim 1, further comprising:
a light reception unit configured to capture an image of the transmitted light appearing on a surface of the target site of the living body.

8. The blood vessel visualization apparatus according to claim 7, further comprising:
a display unit configured to display the image captured by the light reception unit.

9. The blood vessel visualization apparatus according to claim 7, wherein:
the irradiation unit and the light reception unit are configured to face each other with the target site of the living body interposed therebetween.

10. The blood vessel visualization apparatus according to claim 1, wherein:
the fixing portion is formed in a shape that is elongated in a direction crossing the target site of the living body, three or more of the light sources are arrayed along a longitudinal axis of the fixing portion, and a light distribution angle of an outer one of the three or more light sources is larger than a light distribution angle of a centermost one of the three or more light sources.

11. The blood vessel visualization apparatus according to claim 1, further comprising a light emitter configured to convert the transmitted light appearing on a surface of the target site of the living body into light of a different wavelength and to project the light.

12. The blood vessel visualization apparatus according to claim 1, wherein the light source comprises a protrusion portion at a portion that abuts on the skin of the living body.

13. The blood vessel visualization apparatus according to claim 12, wherein the protrusion portion is made of a soft material that is light-transmissive.

14. The blood vessel visualization apparatus according to claim 1, wherein the light source is configured to emit near infrared light.

15. The blood vessel visualization apparatus according to claim 14, wherein the light sources are configured to emit near infrared light having a wavelength of 700 nm or more and 2500 nm or less.

16. A blood vessel puncture system comprising:
a blood vessel visualization apparatus for visualizing a blood vessel inside a living body with light that has been transmitted through the living body, the blood vessel visualization apparatus comprising:
an irradiation unit configured to emit light from a contact portion with skin of the living body, the irradiation unit comprising:
a fixing portion configured to be fixed to a target site of the living body, wherein the fixing portion includes a surface configured to contact the skin of the living body, and
a plurality of light sources located in a light source arrangement area at the surface of the fixing portion that is configured to contact the skin of the living body, wherein:
a brightness and/or a light distribution angle of respective ones of the light sources differ according to positions of the light sources in the light source arrangement area; and
a puncture needle or a catheter assembly.

17. A method for visualizing a blood vessel inside a living body with light that has been transmitted through the living body, the method comprising:
providing a blood vessel visualization apparatus comprising:
an irradiation unit configured to emit light from a contact portion with skin of the living body, the irradiation unit comprising:
a fixing portion configured to be fixed to a target site of the living body, wherein the fixing portion includes a surface configured to contact the skin of the living body, and
a plurality of light sources located in a light source arrangement area at the surface of the fixing portion that is configured to contact the skin of the living body, wherein:
a brightness and/or a light distribution angle of respective ones of the light sources differ according to positions of the light sources in the light source arrangement area;
providing a light reception unit configured to capture an image of the transmitted light appearing on a surface of the target site of the living body;
providing a display unit configured to display the image captured by the light reception unit;
fixing the fixing portion to the target site of the living body; and
viewing the image captured by the light reception unit on the display, and puncturing a blood vessel in the target site of the living body using a puncture needle or a catheter assembly.
